(19) United States
(12) Patent Application Publication
(10) Pub. No.: US 2017/0239098 A1
(43) Pub. Date: Aug. 24, 2017

(54) FOOT BANDAGE FOR COMPRESSION THERAPY OF LYMPHEDEMA

(71) Applicant: Julius Zorn GmbH, Aichach (DE)

(72) Inventor: Uwe SCHETTLER, Aichach (DE)

(73) Assignee: Julius Zorn GmbH, Aichach (DE)

(21) Appl. No.: 15/439,137

(22) Filed: Feb. 22, 2017

(30) Foreign Application Priority Data
Feb. 23, 2016 (DE) 20 2016 100 937.5

(51) Int. Cl. A61F 13/06; A61F 13/00

(52) U.S. Cl. CPC A61F 13/064; A61F 13/00038; A61F 2013/0028

(57) ABSTRACT

In a foot bandage for compression therapy of lymphedema on the foot, with a sole section, side sections connected laterally to sole section, which are joined to each other at least in a section that comes to lie above the heel when the foot bandage is applied, and at least a distal tab section made of an elastic material, which can be wound around the instep and fastened to a side section in order to exert compression on the foot, in which the distal tab section or each tab section has at least one pull tab on which fasteners are arranged, a proximal compression band made from elastic material fastenable removably to the side sections is provided. The compression band has at least a first fastener and is attached to the side sections by means of the first fastener above the heel, when the foot bandage is applied in area.

### FIELD OF THE INVENTION

The invention relates to a foot bandage for compression therapy of lymphedema.

### BACKGROUND

A foot bandage for compression therapy of lymphedema is known from WO 2005/092401 A1 (See, for example FIG. 6 of this PCT application). It includes an inner tube- or sock-like part made of an elastic material (cotton-Lycra blend) as well as three outer bands made of elastic material. The tube- or sock-like part has a sole section and side sections connected laterally to it in one piece, which enclose the metatarsus in the form of a tube when the bandage is positioned. Two or three elastic bands are wound around the instep and fixed there under tension by fasteners to exert compression on the foot. The third band is wrapped around the distal calf area, proximally connected to the foot and also fastened there under tension by fasteners. Owing to tensile stress of the bands, compression is exerted on the foot, especially in the metatarsal area and on the distal section of the lower leg. Lymphedema (fluid accumulations in the intercellular space caused by mechanical insufficiency of the lymphatic system of the human or animal body) can be treated by such compression, by supporting the transport of lymph due to the compression pressure exerted by the foot bandage and thereby reducing lymph congestion.

The foot bandage for compression therapy of lymphedema known from the prior art has the drawback that the arrangement of bands on the tube- or sock-like part is predetermined. Adjustment of the foot band as to the anatomy of the foot of the patient is therefore not possible.

### SUMMARY

The disclosure includes modification of a generic foot bandage so that individual adjustment to the anatomy of the foot of the patient is made possible.

Preferred embodiments of the foot bandage are also disclosed.

The foot bandage according to the invention includes, like the foot bandage of the prior art just described, a sole section as well as two side sections connected laterally to the sole section and a distal tab section made of elastic material with at least one pull tab, which is wound around the instep and fastened to one of the side sections when the bandage is applied in order to exert compression on the foot. The distal tab section then includes at least one pull tab, which can also be designed as an elastic band, as in the known foot bandage, fasteners being arranged on a free end of the pull tab or band with which the pull tab or the band can be fastened under tension to a side section. The foot bandage according to the invention also includes a proximal compression band made of an elastic material, which has a first fastener and can be removably fastened to the side sections of the foot bandage. When the foot bandage is applied, the proximal compression band is releasably fastened to the side sections in an area above the heel by means of the first fastener. Due to the of releasable fastening of the proximal compression band to the side sections of the foot bandage in an area above the heel it is possible to fasten the proximal compression band in the longitudinal direction of the lower leg (in the vertical direction) at different sites of a distal section of the lower leg and thereby adapt the fastening position of the proximal compression band to the anatomy of a patient.

The proximal compression band has two fasteners for fastening of the proximal compression band under tension to a distal section of the lower leg. They are expediently arranged on at least one end section of the proximal compression band. To exert compression pressure, the proximal compression band is wound around a distal section of the lower leg when the foot bandage is applied and fastened there under extension by means of the second fastener. An end section of the proximal compression band is then expediently placed above the other end section of the compression band and fastened to its outside by means of the second fastener.

The fasteners of the pull tabs of the distal tab section as well as the first and second fasteners of the proximal compression band are expediently a hook-and-loop fastener. The hook parts of the hook-and-loop fastener are then preferably removably fastened to the end sections of the corresponding pull tabs or bands. This makes it possible to use the foot bandage for both the left and right foot.

In a preferred embodiment, each side section of the foot bandage according to the invention has a distal tab section, each tab section having at least one pull tab or a band. This permits use of the foot bandage both for a right and left foot. Each tab section preferably has a first and at least a second pull tab. The pull tabs of each tab section are then expediently made from the same elastic material as the proximal compression band. The side sections of the sole section are also preferably made from the same material, which is preferably an elastic textile material with a maximum elongation in the range of 15 to 50% of the unstretched length. The textile material, for example, can be a textile fabric made of polyamide and elastane, especially with 87% polyamide and 13% spandex, relative to the weight of the textile fabric.

If a distal tab section has several pull tabs, adjacent pull tabs of a tab section are expediently separated from each other by an indentation in the corresponding tab section. It is then possible to fasten the pull tabs of a tab section at an angle to each other on the opposite tab section when the foot bandage is applied. Adjustment of the foot bandage to the anatomy of the patient can also occur on this account.

At least each pull tab of one of the two distal tab sections also expediently has a fastener with which the pull tab can be releasably fastened to the opposite side section or its tab section. The fastener can be designed as a hook part of the hook-and-loop fastener and removably arranged on the pull tab of one of the two tab sections. This makes it possible to remove the hook parts of the hook-and-loop fastener from the pull tabs of one tab section and fasten them to the pull tabs of the other tab section. The foot bandage can then be used for both a right and left foot.

In a preferred embodiment example the two side sections and the sole section of the foot bandage are formed by separate parts that are fastened, especially stitched together. The side sections in the area of the heel are then joined together along a vertical seam, the vertical seam lying in the area of the Achilles tendon when the foot bandage is applied. Each side section is also joined, especially stitched, to the sole section along the horizontal seam. Each side section, in a preferred embodiment of the foot bandage according to the invention has a proximal and a distal edge, which are at least essentially straight. A tab section expediently having two or more pull tabs or bands is arranged in each side section between the proximal edge and the distal edge. The extensions of the proximal and distal edge of a side section then meet preferably at an angle lying between 40 and 50° and especially 45°.

Cushioning is expediently incorporated in the sole section to increase wearing comfort. Cushioning can then be sewn especially to the outside of the sole section.

### BRIEF DESCRIPTION OF THE DRAWINGS

These and additional features and advantages of the foot bandage according to the invention are apparent from the embodiment example described below with reference to the accompanying drawings, in which the drawings show:

FIGS. 1a and 1b: Depiction of a foot bandage for compression therapy of lymphedema according to the invention applied to a foot in a side view, the foot bandage including a removable proximal compression band, which is shown in the open state in the depiction of FIG. 1a and in the closed state in the depiction of FIG. 1b;

FIGS. 2a to 2k: Schematic depiction of the steps to position the foot bandage of FIG. 1 on a foot of a patient.

### DETAILED DESCRIPTION

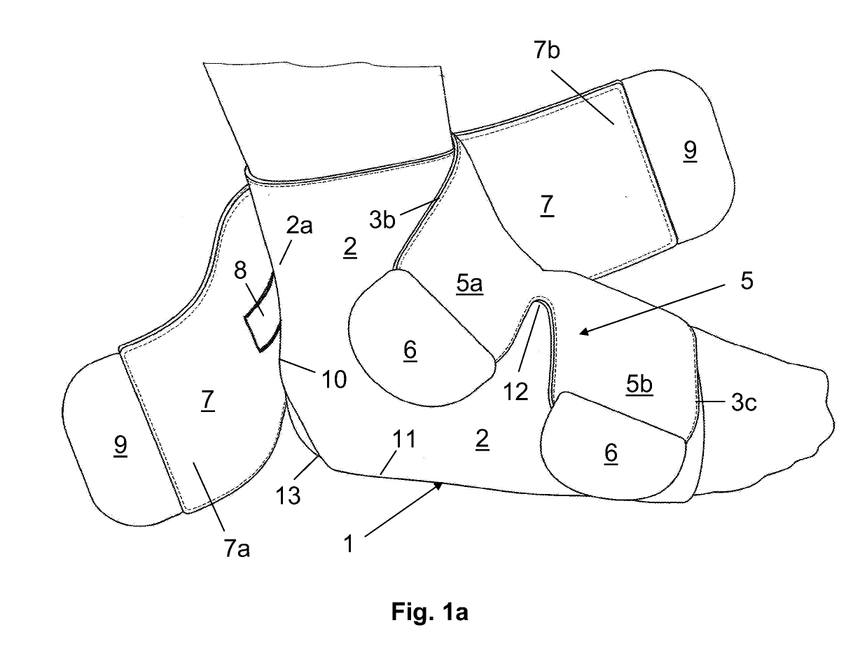

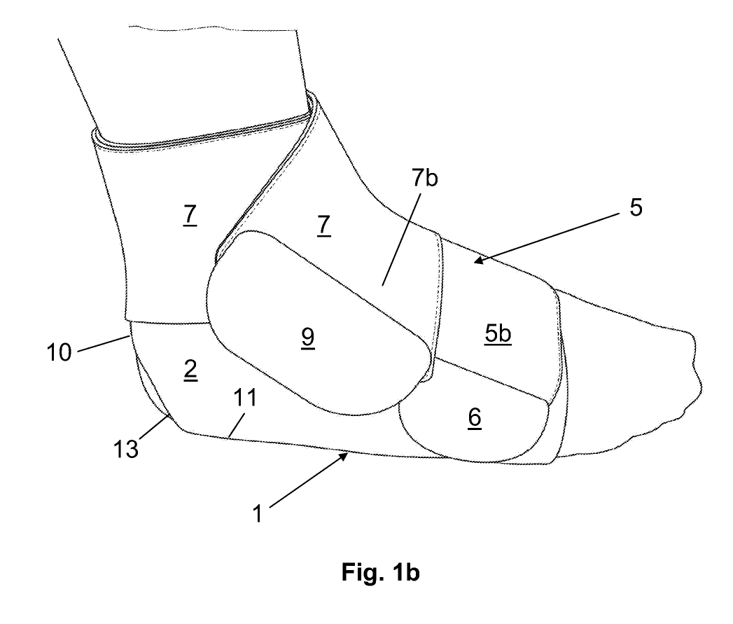

The foot bandage depicted in FIGS. 1a and 1b includes the sole section 1 and side sections 2, 3 connected laterally to the sole section 1. Each side section 2, 3 is then expediently joined, especially stitched, to the sole section 1 along the horizontal seam 11. However, it is also possible to form the sole section 1 and the side sections connected to it in one piece and therefore seamlessly.

The two side sections 2, 3 are joined to each other by a vertical seam 10 in an area 2a, 3a above the heel. When the foot bandage is applied, the vertical seam 10 runs in the vertical direction above the heel in the area of the Achilles tendon of the patient. The foot bandage has a recess 13 in the heel area. Cushioning is preferably incorporated in the sole section 1 in order to increase wearing comfort. The cushioning can be sewn, for example, onto an outside of the sole section 1.

Each side section 2, 3 has a proximal edge 2b, 3b and a distal edge 2c, 3c. A tab section 4, 5 is arranged in each side section 2, 3 between the proximal edge 2b, 3b and the distal edge 2c, 3c. The extensions of the proximal edge 2b, 3b and the distal edge 2c, 3c then meet at an angle between 40 and 50° and preferably at an angle of 45°.

The sole section 1, the two side sections 2, 3 and each distal tab section 4, 5 are then expediently made from an elastic material, for example, an elastic short-pull textile material. The elastic material expediently has a maximum range of elongation between 15 and 50% of the unstretched state. The elastic material can be a textile fabric made of polyamide and spandex, for example, with 87% polyamide and 13% spandex, relative to the weight of the textile fabric. The maximum elongation of the elastic material preferably lies in the range from 15 to 50% of the unstretched length and with particular preference either between 15 and 20% or between 25 and 35%.

Each distal tab section 4, 5 includes in the embodiment example depicted in the drawing two pull tabs 4a, 4b and 5a, 5b. The pull tabs 4a, 4b and 5a, 5b of a tab section 4, 5 are separated from each other by an indentation 12 in the corresponding tab section 4, 5.

Fasteners 6 are arranged on at least one of the two distal tab sections 4, 5 for fastening of the foot bandage to a foot of a patient. These fasteners 6 are expediently a hook-and-loop fastener. The fasteners 6 in the embodiment example depicted in the drawing are removably arranged on a free end of the two pull tabs 5a, 5b of a tab section 5.

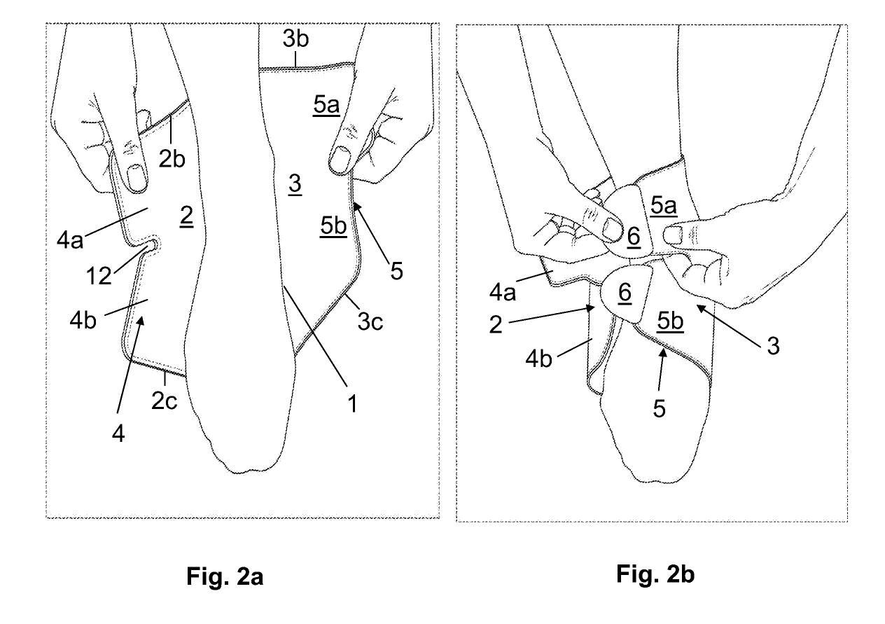

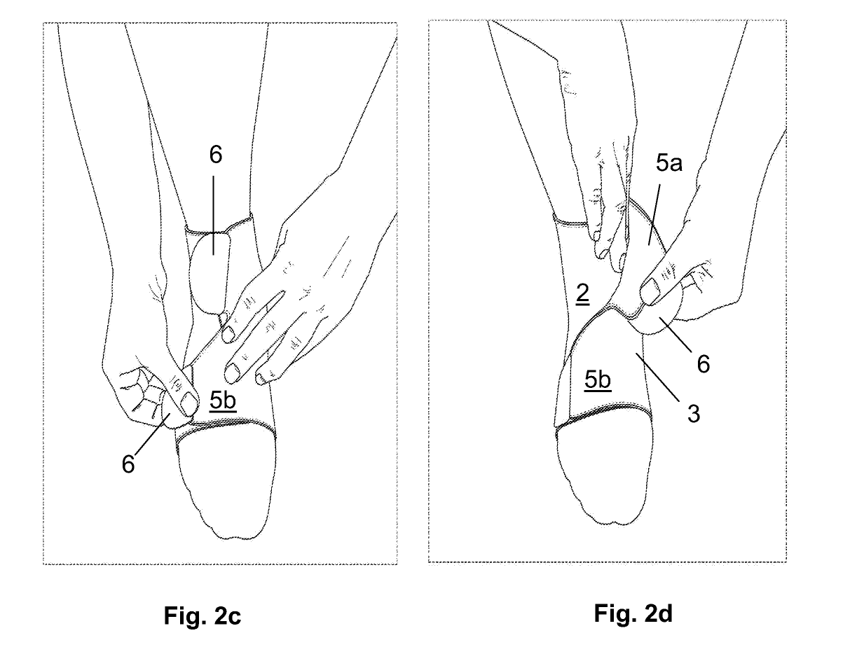

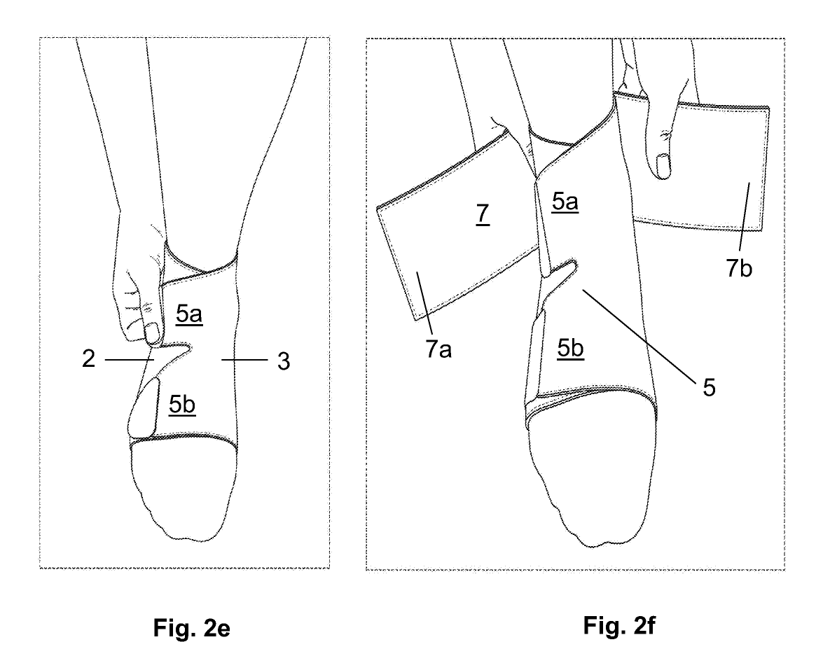

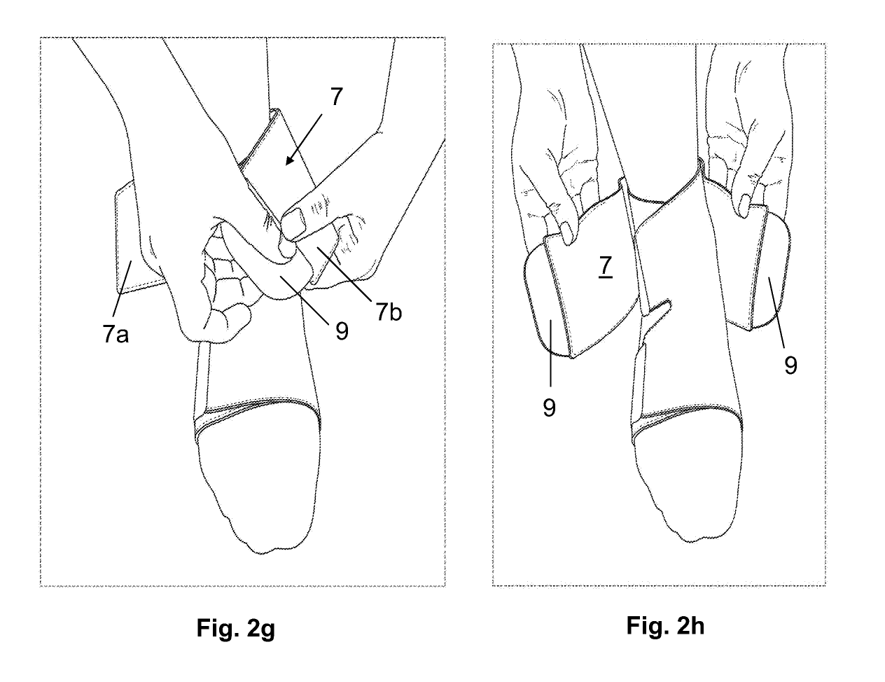

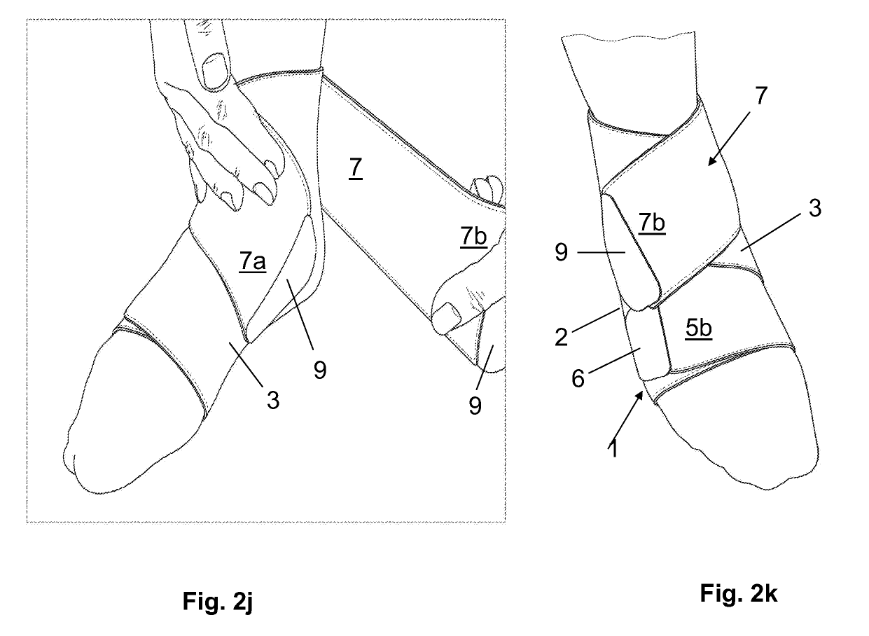

The part of the foot bandage consisting of the sole section 1 and the side sections 2 and 3 with the distal tab sections 4, 5 can be fastened under tension to the foot of the patient by means of fastener 6 so that this part of the foot bandage exerts a compression pressure on the foot. The compression pressure exerted on the foot when the foot bandage is applied, depending on the set elongation, expediently lies in the range between 8 and more than 40 mmHg and can be set to compression values up to 60 mmHg. Elongation of the elastic material then occurs by manual tension on the distal tab sections 4, 5 and is fixed by attaching fastener 6. For this purpose, the distal tab sections 4, 5, on which the pull tabs 4a, 4b and 5a, 5b are arranged are wound around the instep, and the distal tab section 5, on the pull tabs 5a, 5b of which the fasteners 6 are arranged, is fastened under tension by means of fastener 6 to the opposite side section 2. Positioning of the foot bandage on a foot is explained below in further detail with reference to FIGS. 2a to 2k.

The foot bandage according to the invention also includes a removable proximal compression band 7. This compression band 7 is shown in the open state in the depiction of FIG. 1a and in the closed state in the depiction of FIG. 1b. The proximal compression band 7 is removably fastened to side sections 2, 3 when the foot bandage is applied. Like the sole section 1 and the side sections 2 and 3 with the distal tab sections 4, 5, the proximal compression band 7 is also expediently formed from an elastic material, i.e., from the same elastic material as a sole section and the two side sections with the tab sections. For removable fastening of the proximal compression band 7 to the side sections 2, 3 the proximal compression band has a first fastener 8. The first fastener 8 is expediently formed by a hook part of a hook-and-loop fastener, which is fastened to a central location, especially stitched, on the inside of compression band 7. The first fastener 8 is apparent in the depiction of FIG. 1a. The proximal compression band 7 is fastened by means of the first fastener 8 in the area 2a, 3a above the heel on the outside of side sections 2, 3. To exert a compression pressure, the proximal compression band 7 is wrapped around a section of the lower leg of the patient and fastened there by means of a second fastener 9 under elongation of the proximal compression band. The proximal compression band 7 then has a first end section 7a and a second end section 7b, second fasteners 9 being provided on at least one of the two end sections 7a. The second fasteners 9 are expediently a hook-and-loop fastener and especially a hook part of the hook-and-loop fastener removably fastened to end section 7a.

Positioning of the foot bandage on the foot of a patient is explained below with reference to FIGS. 2a to 2k. For this purpose, the part of the foot bandage consisting of the sole section 1 and the two side sections 2 and 3 with the distal tab sections 4, 5 is initially positioned on the foot of the patient, as shown in FIG. 2a, the sole section 1 coming to lie in the area of the sole of the foot and the two side sections 2, 3 with the distal tab sections 4, 5 arranged on them being wound around the instep. The vertical seam 10, through which the two side sections 2, 3 are joined to each other, then comes to lie in the area of the Achilles tendon and the vertical seam 10 runs essentially in the vertical direction. The recess 13 lies in the area of the heel of the patient.

By manual tension on the pull tabs 4a, 4b and 5a, 5b of the distal tab sections 4, 5 compression is exerted on the foot of the patient. This compression is fixed by fastening the pulled tab sections 4, 5 on the opposite side sections 2, 3. For this purpose, the fasteners 6 arranged on the free ends of pull tabs 5a, 5b of a tab section 5 are fixed on the outside of the opposite side section 2. The fasteners 6 are then expediently designed to be removable. This makes it possible to use the foot bandage for both the right and left foot. Fastening of the fastener 6 to the free ends of pull tabs 5a, 5b of a distal tab section 5 is shown in FIG. 2b. The fasteners 6 are hook-and-loop fasteners, whose hook part is joined to one section on the outside of the free end of the pull tabs 5a, 5b. Another section of the fastener 6 then remains free so that it can be joined to the outside of the opposite side section 2.

For fixation of the foot bandage on the foot, a first pull tab 5b and then the second pull tab 5a of a distal tab section 5 are initially attached by fastener 6 to the outside of the opposite side section 2, as shown in FIGS. 2c, 2d and 2e. Tension is then exerted on the tab section 5 via pull tabs 5a, 5b, which is transferred to the side section 3 and the sole section 1. By attaching the fastener 6 of the pull tabs 5a, 5b to the opposite side section 2, the elongation produced by tension on the elastic material is fixed so that compression is exerted on the foot of the patient.

The proximal compression band 7 is then fastened in the area of the lower leg of the patient. For this purpose, the inside of the proximal compression band 7 is initially fastened by means of the first fastener 8 to the outside of side sections 2, 3. Fastening then occurs in the area 2a, 3a of side sections 2, 3 that lies above the heel. Fastening of the proximal compression band 7 to the side sections 2, 3 via the first fastener 8 is shown in FIG. 2f. The precise position of the proximal compression band 7 can then be correspondingly adjusted to the individual requirements of compression therapy, in which case the proximal compression band 7 can be pushed in the proximal-distal direction. The proximal compression band 7 can therefore be positioned and fastened in the section of the lower leg or also in the area of the ankle joint of the patient.

For fastening of the proximal compression band 7 to a desired location on the lower leg or ankle joint of the patient, removable second fasteners 9 are initially arranged on one of the end sections 7a of the proximal compression band 7, as shown in FIG. 2g. The second fasteners 9 are expediently hook parts of a hook-and-loop fastener, which are firmly joined to the outside of the proximal compression band 7 in the area of one of the end sections 7a. A second fastener 9 is also expediently arranged, as shown in FIG. 2h, on the other end section 7b of the proximal compression band 7. The proximal compression band 7, as shown in FIG. 2g, is then fastened by means of the second fastener 9 initially to an end section 7a on the outside of the side section 3 of the foot bandage. The other end section 7b of the proximal compression band 7 is then also fastened to the opposite side section 2 of the foot bandage by means of the second fastener 9. The proximal compression band 7 is then manually held under tension and this tension is fixed by attaching the second fastener 9 so that the proximal compression band 7 also exerts compression on the lower leg or foot of the patient in the area of the ankle joint. The positioned foot bandage in a top view of the foot of the patient is shown in FIG. 2k.

When the tab sections 4, 5 are fastened to the opposite side sections 3 and 2, it must be kept in mind that the foot of the patient is fully enclosed by the side sections 2 and 3 and tab sections 4, 5, so that no free locations form through which body tissue might swell. The proximal compression band 7 should also be correspondingly positioned so that it at least partially overlaps the side sections 2, 3.

The invention is not restricted to the embodiment depicted in the drawings. For example, the number of pull tabs of the tab sections 4, 5 can be adjusted to the size of the bandage and the anatomy of the foot. In longer bandages, each tab section 4, 5 can also include three or more pull tabs; and in shorter bandages, only one pull tab can be sufficient. It is also sufficient if a tab section with at least one pull tab is arranged only on one of the two side parts 2, 3. The embodiment of the invention with tab sections 4, 5 on both side parts 2, 3 depicted here, however, has the advantage that the foot bandage can be used for both a right and left foot.

The elongation characteristics of the elastic materials are variable over a wide range and can be adjusted to the requirements of the compression treatment to be performed with the bandage. Other fasteners instead of hook-and-loop fasteners can also be used, such as press studs or hook-and-eye closures.

What is claimed is:

1. A foot bandage for compression therapy of lymphedema on the foot with a sole section, side sections laterally connected to sole section, which are joined to each other at least in back sections that come to lie above the heel when the foot bandage is positioned, and at least one distal tab section made from an elastic material, which can be wound around the instep and fastened to a side section in order to exert compression on the foot, in which the or each distal tab section has at least one pull tab on which fasteners are arranged, wherein a proximal compression band that can be removably fastened to the side sections made from elastic material, in which the compression band has first fasteners and is fastened to the side sections by the first fasteners in the area above the heel, when the foot bandage is applied.

2. The foot bandage according to claim 1, wherein the proximal compression band has a first end section and a second end section, as well as at least a second fastener arranged on one of the end sections and is wound around a distal section of the lower leg and fastened there under elongation by means of the second fastener, when the foot bandage is applied.

3. The foot bandage according to claim 1, wherein a first side section and a second side section, the first side section having a first distal tab section and the second side section having a second distal tab section.

4. The foot bandage according to claim 3, wherein the first distal tab section, the second distal tab section each has a first pull tab and at least a second pull tab.

5. The foot bandage according to claim 4, wherein a fastener for releasable fastening to the opposite side section is arranged on each pull tab of the first distal tab section and/or on each pull tab of the second distal tab section.

6. The foot bandage according to claim 3, wherein the fasteners are releasably arranged on the first distal tab section and the second distal tab section.

7. The foot bandage according to claim 4, wherein the fasteners are releasably arranged on the first pull tab and the second pull tab.

8. The foot bandage according to claim 3, wherein the adjacent pull tabs of the first distal tab section and/or the second distal tab section are separated from each other by an indentation in the corresponding tab section.

9. The foot bandage according to claim 1, wherein the side sections are made from the same elastic material as the tab section or each distal tab section.

10. The foot bandage according to claim 1, wherein the distal tab section or each distal tab section is formed in one piece on one of the side sections.

11. The foot bandage according to claim 1, wherein the side sections are joined to each other along a vertical seam, the vertical seam running in the area of the Achilles tendon, when the foot bandage is applied.

12. The foot bandage according to claim 1, wherein each side section is joined to the sole section along a horizontal seam.

13. The foot bandage according to claim 1, wherein each side section has a proximal edge and a distal edge and that a distal tab section is arranged between the proximal edge and the distal edge.

14. The foot bandage according to claim 13, wherein the extensions of the proximal edge and the distal edge of each side section meet at an angle from 40 to 50° and especially at a 45° angle.

15. The foot bandage according to claim 1, wherein a recess is provided in the heel area.

16. The foot bandage according to claim 1, wherein cushioning is incorporated in the sole section.

17. The foot bandage according to claim 16, wherein the cushioning is sewn to an outside of the sole section.

18. The foot bandage according to claim 1, wherein the fasteners are hook-and-loop fasteners, the hook part of the hook-and-loop fastener being removably fastened to the pull tabs and/or a proximal compression band.

19. The foot bandage according to claim 1, wherein the proximal compression band can be attached by means of the first fastener in the vertical direction on different locations in the area of section above the heel on side sections.

20. The foot bandage according to claim 1, wherein the two side sections are joined to each other along a vertical seam and the proximal compression band is releasably fastened to the outside of the two side sections, when the foot bandage is applied in the area of the vertical seam.
